(19) United States
(12) Patent Application Publication
(10) Pub. No.: US 2023/0273200 A1
(43) Pub. Date: Aug. 31, 2023

(54) ELECTROCHEMICAL IMMUNOSENSOR FOR DETECTION OF CALPROTECTIN

(71) Applicant: Eli Lilly and Company, Indianapolis, IN (US)

(72) Inventors: Muhammad Ashraful ALAM, West Lafayette, IN (US); Marco FRATUS, West Lafayette, IN (US); Rahim RAHIMI, West Lafayette, IN (US); Vidhya SELVAMANI, West Lafayette, IN (US); Mohit Singh VERMA, West Lafayette, IN (US); Jiangshan WANG, West Lafayette, IN (US)

(21) Appl. No.: 18/005,779

(22) PCT Filed: Aug. 11, 2021
(86) PCT No.: PCT/US2021/045470

§ 371 Date: Jan. 17, 2023

Related U.S. Application Data
(60) Provisional application No. 63/064,963, filed on Aug. 13, 2020.

(51) Int. Cl. G01N 33/543

(52) U.S. Cl. CPC G01N 33/5438; G01N 2333/4727

(57) ABSTRACT

An electrochemical biosensor for detection of a calprotectin antigen in a sample solution. The electrochemical biosensor includes a reference electrode, a counter electrode, and a working electrode. The working electrode includes a surface coated with an anti-calprotectin antibody that binds to the calprotectin antigen in the sample solution. The electrochemical biosensor detects the concentration of the calprotectin antigen in the sample solution based on a resistance change at the surface of the working electrode.

### FIELD OF THE DISCLOSURE

The present disclosure pertains to immunosensors, and, in particular, to a disposable immunosensor that can detect a calprotectin antigen as a biomarker in a sample solution for diagnosis of Inflammatory Bowel Diseases (IBD).

### BACKGROUND

IBD is characterized by chronic inflammation in a gastrointestinal tract with highest prevalence in Europe and North America. By 2015, the Center for Disease Control (CDC) reported that about 1.3% of U.S. adults are diagnosed with IBD. Crohn’s disease (CD) and ulcerative colitis (UC) are two common causes of IBD, and the symptoms are abdominal pain, diarrhea, and bloody stool. IBD is caused by uncontrolled immune response and the risk is high in a genetically susceptible individual. The incidence of IBD is more common in the 20’s to 40’s of the adulthood. IBD treatments are tailored to treat active diseases, maintain remission and reduce the risk of relapse. Hence, it may be desirable to continuously monitor IBD patients. The standard procedure for evaluating intestinal inflammation and mucosal healing is by imaging techniques such as endoscopy and colonoscopy. Based on the endoscopic images, the phase (active and remission) and level of inflammation throughout the GI tract is confirmed. These procedures are invasive, time consuming and may cause intestinal perforation. It is also reported that IBD symptoms are worsened by these invasive techniques and could be burdensome on patients. Therefore, it may be desirable to provide a non-invasive detection method to identify IBD.

Many biomarkers have been evaluated as a non-invasive approach to facilitate IBD diagnosis, reduce cost, and decrease patient discomfort as an alternative approach to endoscopy and colonoscopy in diagnosis of IBD. Among different biomarkers, calprotectin (CP) level in feces and serum has demonstrated an association with the degree of inflammation. Various CP detection platforms have been developed which include enzyme-linked immunosorbent assay (ELISA), as well as point of care test (POCT) kits. Despite the high sensitivity and robustness, ELISA displays shortcomings including longer processing time, multiple washing steps, expensive chemicals for labels, and trained professionals required for operation. Alternatively, the currently available POCT kits are based on techniques such as immunochromatographic assay and turbidimetric immunoassay, which makes the POCT faster, however, visual readouts from these assays are not always reliable and the POCT tests are not always accurate.

### SUMMARY

According to an embodiment of the present disclosure, an electrochemical biosensor for detection of a calprotectin antigen in a sample solution is provided. The electrochemical biosensor includes a reference electrode, a counter electrode, and a working electrode. The working electrode includes a surface that has been coated with an anti-calprotectin antibody where the anti-calprotectin antibody binds to the calprotectin antigen in the sample solution. The electrochemical biosensor can detect a concentration of the calprotectin antigen in the sample solution based on a resistance change at the surface of the working electrode.

According to another embodiment of the present disclosure, a method of determining a concentration of a calprotectin antigen in a sample solution with a biosensor is provided. The method includes providing the biosensor where the biosensor includes a reference electrode, a counter electrode, and a working electrode. The working electrode has a surface coated with an anti-calprotectin capture antibody. The method also includes providing the sample solution which consists essentially of the calprotectin antigen and lacks an anti-calprotectin detection antibody. The method also includes exposing the sample solution to the biosensor where the calprotectin antigen binds to the anti-calprotectin capture antibody on the working electrode. The method also includes measuring a current between the counter electrode and the working electrode. The method also includes determining the concentration of the calprotectin antigen in the sample solution based upon the measured current.

According to yet another embodiment of the present disclosure, a system for detecting the concentration of a calprotectin antigen in a sample solution is provided. The system includes an electrochemical biosensor where the biosensor includes a working electrode, a reference electrode, and a counter electrode. The outer surface of the working electrode is coated with an anti-calprotectin antibody which binds to the calprotectin antigen in the sample solution. The system also includes an analyser in electrical communication with the biosensor. The analyser can determine a concentration of the calprotectin antigen in the sample solution from 4 ng/mL to 240 ng/mL based upon an electrical current flowing between the working electrode and the counter electrode.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a perspective view of an exemplary electrochemical immunosensor of the present disclosure.

FIG. 2 is a schematic view of a system used to determine the concentration of an analyte in a sample solution using the electrochemical immunosensor of FIG. 1

FIG. 3 is a graph of multiple cyclic voltammograms generated in a second experimental example.

FIG. 4 is a graph of a Nyquist plot generated in a second experimental example.

FIG. 5 is a graph of multiple Nyquist plots generated in a second experimental example.

FIG. 6 is a graph of multiple cyclic voltammograms generated in a third experimental example.

FIG. 7 is a graph of current in relation to antibody concentration generated in a third experimental example.

FIG. 8 is a graph of multiple Nyquist plots generated in a third experimental example.

FIG. 9 is a graph of resistance in relation to analyte concentration attained in a third experimental example.

FIG. 10 is a graph of a change in resistance in relation to changing analyte concentrations generated in a third experimental example.

FIG. 11 is a graph of multiple Nyquist plots generated in a fourth experimental example.

FIG. 12 is a graph of a change in resistance in relation to analyte concentration generated in a fourth experimental example.

FIG. 13 is a graph of multiple Nyquist plots generated in a fifth experimental example.

FIG. 14 is a graph comparing the selectivity of different samples generated in a fifth experimental example.

FIG. 15 is a graph of multiple Nyquist plots generated in a sixth experimental example.

FIG. 16 is a graph of a comparison of the selectivity of different samples generated in a sixth experimental example.

FIG. 17 is graph of a comparison of a concentration range of an ELISA test vs. an immunosensor generate in a seventh experimental example.

Corresponding reference characters indicate corresponding parts throughout the figures. Although the drawings represent embodiments of the present invention, the drawings are not necessarily to scale, and certain features may be exaggerated or omitted in some of the drawings in order to better illustrate and explain the present invention.

### DETAILED DESCRIPTION

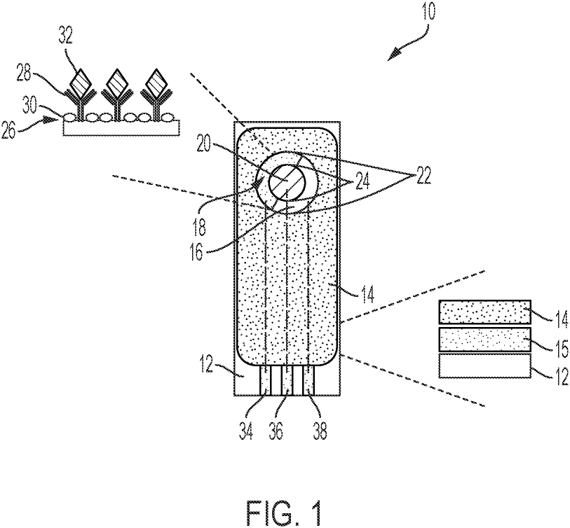

FIG. 1 illustrates an exemplary embodiment of an electrochemical immunosensor 10. Immunosensor 10 can be used in label free detection of a biomarker, such as calprotectin (CP), which is indicative of IBD in a patient. Calprotectin is a heterodimeric zinc and calcium-binding protein composed of S100A8 and S100A9 and is abundantly found in neutrophils (e.g., 40-60% of the cytoplasmic protein in neutrophil is calprotectin). In IBD, neutrophils migrate to the inflammatory site and perform phagocytosis. The neutrophil releases anti-pathogenic proteins and tissue-damaging agents as a part of the innate immune response. Calprotectin is one such antimicrobial protein, which eliminates microbes by nutritional immunity. The calprotectin amount in feces is relative to infiltered neutrophil in the mucosa of gastrointestinal tract (GI). Illustratively, the immunosensor 10 is a miniaturized electrochemical immunosensor that can be included in a POCT kit. This miniaturized configuration allows for fast and convenient identification of the biomarker in a sample taken from the patient (e.g., fecal, serum, etc.) since neither dedicated laboratory analysis (e.g., as in the case of ELISA) nor an invasive medical procedure (e.g., as in the case of traditional imaging techniques) is required.

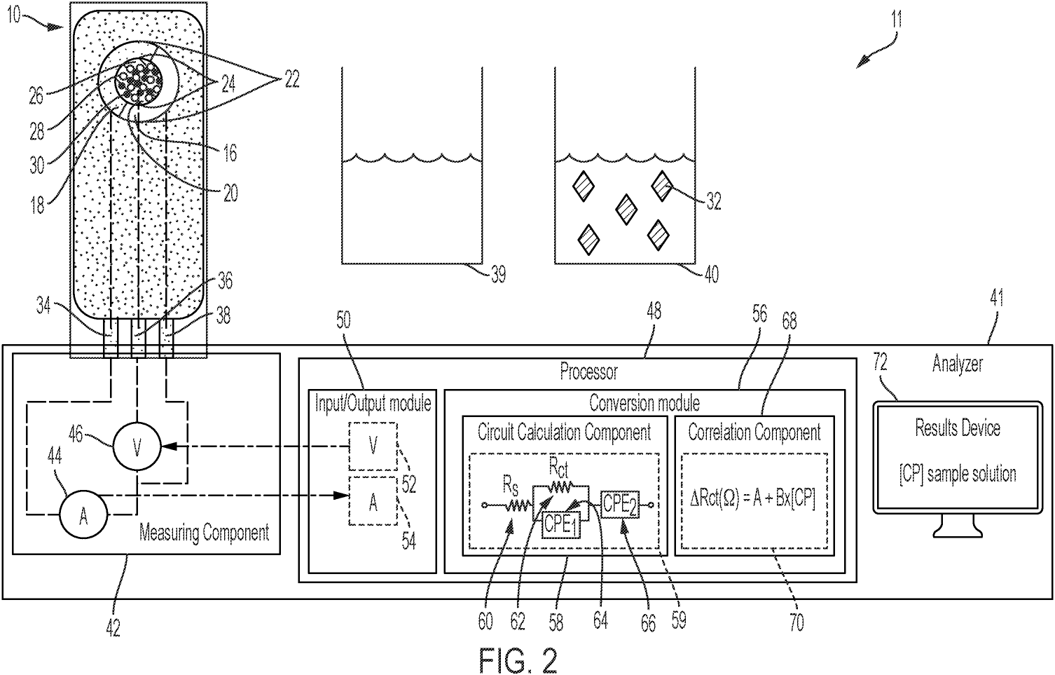

Immunosensor 10 includes a substrate 12, an insulator 14, and an electrode layer 15 including a reference electrode 16, a counter electrode 18, and a working electrode 20. Immunosensor 10 can be fabricated in layers, where the components of immunosensor 10 are formed additively on top of one another. For example, substrate 12 may be the bottom layer of immunosensor 10 and formed from, polymer, glass, or other suitable materials. Each of the electrodes (e.g., including reference electrode 16, counter electrode 18, and working electrode 20) are formed on top of substrate 12 in an additive process. Insulator 14 is formed on top of both substrate 12 and the electrode layer 15 to electrically isolate each of the electrodes from one another. Each of the electrodes includes a corresponding lead (i.e., reference electrode 16 includes lead 34, counter electrode 18 includes lead 38, and working electrode 20 includes lead 36) which are used to connect immunosensor 10 to an analyzer 41, as illustrated in, and discussed in relation to, FIG. 2 below. The analyzer 41 is used to detect a change in the resistance associated with the working electrode 20, which is caused by a reaction of a calprotectin biomarker 32 (e.g., a calprotectin antigen biomarker), in a sample solution (e.g., fecal, serum, etc.) with an anti-calprotectin capture antibody bound to an outer surface 26 of working electrode 20. Such detection is discussed further in relation to FIG. 2 below.

Immunosensor 10 includes aperture 22. Aperture 22 is a hole in insulator 14 that allows for each of the electrodes to contact a sample solution containing the calprotectin biomarker 32. Aperture 22 can be a circular opening that exposes portions of all three electrodes (e.g., including reference electrode 16, counter electrode 18, and working electrode 20). A sample solution containing the calprotectin biomarker 32 can be introduced into aperture 22. Due to the conductance of the liquid sample, electrical current can pass between each of the electrodes through the solution (e.g., an electrical circuit is complete between the electrode by the sample solution). The current passing between the counter electrode 18 and the working electrode 20 can be used to determine the concentration of the calprotectin biomarker 32 in the sample solution based upon an electrochemical analysis of the sample solution, as discussed further in relation to FIG. 2 below.

Reference electrode 16 serves as the grounding electrode for immunosensor 10. Reference electrode 16 can be formed from a conductive metallic material such as silver or a silver compound (e.g., silver chloride). Reference electrode 16 surrounds a portion of working electrode 20 exposed through aperture 22 and extends longitudinally towards lead 38 beneath insulator 14. Reference electrode 16 is connected to a constant voltage source by lead 38 and provides a constant voltage between reference electrode 16 and working electrode 20. This voltage is used as the reference voltage during the electrochemical analysis of the sample solution, as discussed further in relation to FIG. 2 below.

Counter electrode 18 is the auxiliary electrode for immunosensor 10. Counter electrode 18 can be formed from a conductive metallic material such as platinum. Counter electrode 18 also surrounds an opposing portion of working electrode 20 exposed through aperture 22 across from reference electrode 16 and extends longitudinally towards lead 34 beneath insulator 14. Counter electrode 18 is used in combination with working electrode 20 to measure the current flowing between the two electrodes during the electrochemical analysis of the sample solution, as discussed further in relation to FIG. 2 below.

Working electrode 20 serves as the reaction site 24 for immunosensor 10. More specifically, the area of the working electrode 20 that is exposed through aperture 22 serves as the reaction site 24 where the anti-calprotectin capture antibody 28 and the calprotectin biomarker 32 interact. The extent of the reaction at the reaction site 24 (e.g., as measured by the current flowing between the counter electrode 18 and the working electrode 20 during the electrochemical analysis of the sample solution), is used to determine the concentration of the calprotectin biomarker 32 in the sample solution. Working electrode 20 can be formed from a variety of materials and by a variety of methods, such as a gold electrode formed by screen printing (e.g., a screen-printed gold electrode (SPGE)). These materials and methods enable binding of antibodies, such as an anti-calprotectin capture antibody 28, to an outer surface 26 of working electrode 20, illustratively an upper surface of working electrode 20. The anti-calprotectin capture antibody 28 reacts (e.g., binds with) the calprotectin biomarker 32 present in the sample solution. The reaction of the calprotectin biomarker 32 and the anti-calprotectin capture antibody 28 causes a change in the current passing between the working electrode 20 and the counter electrode 18 based upon a resistance change at the outer surface 26 of working electrode 20. The change in current can be used to calculate the concentration of the calprotectin biomarker 32 in the sample solution during the electrochemical analysis of the sample solution, as discussed further in relation to FIG. 2 below.

The outer surface 26 of working electrode 20 is functionalized by coating the outer surface 26 with the anti-calprotectin capture antibody 28. In this case, a thiolated (e.g., directionalized) form of anti-calprotectin capture antibody 28 is functionalized to the outer surface 26 of working electrode 20. Coating the outer surface 26 of working electrode 20 with thiolated anti-calprotectin capture antibodies 28 provides a functional, high-affinity surface for the binding (e.g., reaction) of the anti-calprotectin capture antibody 28 with the calprotectin biomarker 32 by the upright orientation of the thiolated anti-calprotectin capture antibodies 28 on the outer surface 26 of the working electrode 20. After functionalization with the anti-calprotectin capture antibody 28, a blocking agent 30 is also fixed to the outer surface 26 of working electrode 20 to prevent non-site specific binding of the anti-calprotectin capture antibody 28 with calprotectin biomarker 32.

For example, an anti-calprotectin antibody (such as a mouse monoclonal anti-calprotectin antibody) may be used as the anti-calprotectin capture antibody 28. In this case, a solution of a thiolation reagent is mixed with the anti-calprotectin antibody and incubated, which forms a thiolated anti-calprotectin antibody and the thiolation reagent modifies some amines to sulfhydryl, which forms thioether linkages. The thiolated calprotectin antibodies are then coated onto the outer surface 26 of working electrode 20 by drop casting the anti-calprotectin antibody solution on the working electrode 20 and incubating the working electrode 20. The working electrode 20 is then washed with a buffer solution (e.g., phosphate-buffered saline (PBS)) to remove excess calprotectin antibodies (e.g., excess anti-calprotectin capture antibodies 28). The blocking agent 30, such as bovine serum albumin (BSA), is added to the outer surface 26 of the working electrode 20 to block non-site specific binding of the calprotectin biomarker 32 with the anti-calprotectin antibody.

The concentration of the anti-calprotectin antibody 28 in the anti-calprotectin antibody solution can be 1 µg/mL to 20 µg/mL. More specifically, the concentration of the anti-calprotectin antibody 28 in the anti-calprotectin antibody solution can be 2 µg/mL to 10 µg/mL, 4 µg/mL to 6 µg/mL, 4.5 µg/mL to 5.5 µg/mL, 4.9 µg/mL to 5.1 µg/mL, or 5 µg/mL. The concentration of the anti-calprotectin antibody solution should allow for the appropriate amount of thiolated calprotectin antibodies 28 to be fixed to the outer surface 26 of the working electrode 20 without overcrowding the surface 26 of the working electrode 20. In the present case, functionalization with the 5 µg/mL antibody solution resulted in the widest range in detectability during the electrochemical analysis performed on the sample solution 40.

As described above, a sample solution 40 containing the calprotectin biomarker 32 is introduced into aperture 22, which completes a circuit between the electrodes. In this case, a reaction takes place at reaction site 24. The reaction of the calprotectin biomarker 32 and the anti-calprotectin capture antibody 28 can cause a resistance change at the outer surface 26 of the working electrode 20, which can be detected by the analyzer 41 as discussed in relation to FIG. 2 herein.

FIG. 2 illustrates a system 11 for detecting the concentration of calprotectin biomarker 32 in a sample solution 40 using the above-described immunosensor 10. The system 11 includes the immunosensor 10, a calibration solution 39 (e.g., potassium ferricyanide) that lacks any calprotectin biomarker 32, the sample solution 40 including the calprotectin biomarker 32, and an analyzer 41 including a measuring component 42, a processor 48, and results device 72.

As described in relation to FIG. 1 above, the immunosensor 10 includes reference electrode 16, counter electrode 18, and working electrode 20, where the outer surface 26 of the working electrode 20 has been functionalized with the anti-calprotectin capture antibody 28 (e.g., an anti-calprotectin antibody), and the blocking agent 30 has been added to the working electrode 20 to prohibit non-site specific binding of the calprotectin biomarker 32. In use, the calibration solution 39 is introduced onto the immunosensor 10 to perform a calibration measurement using the analyzer 41. Then, the sample solution 40 containing the calprotectin biomarker 32 is introduced onto immunosensor 10 to perform an actual measurement of sample solution 40 using the analyzer 41. The sample solution 40 can include a fecal sample, a serum sample, or any other suitable sample that contains the calprotectin biomarker 32. The sample solution 40 may be prepared by diluting a raw sample (e.g., a raw fecal sample). The calibration solution 39 and the sample solution 40 may each be placed into the aperture 22 of immunosensor 10 (e.g., via a dropper, pipet, dipping immunosensor 10 into solutions 39 and/or 40, or other suitable means).

In the case of the sample solution 40, the calprotectin biomarker 32 binds with the anti-calprotectin capture antibody 28 at the reaction site 24. This reaction causes a buildup of electrical resistance (e.g., a resistance to current flowing between the counter electrode 18 and the working electrode 20) at the outer surface 26 of the working electrode 20. This resistance is attributed to the accumulation of biomolecules (e.g., bound calprotectin biomarker 32 with anti-calprotectin capture antibody 28) at the outer surface 26 of the working electrode 20. This accumulation serves as an insulating layer that builds on the working electrode 20 and limits transfer of ions to the working electrode 20. For example, the bound calprotectin biomarker 32 on the functionalized calprotectin antibodies 28 act as an inert insulating layer which prevents the current from reaching the outer surface 26 of the working electrode 20. This phenomena is known as charge transfer resistance (R_{CT}), where such resistance was found to increase linearly in relation to the concentration of calprotectin biomarkers 32 in the sample solution 40.

The use of a resistance (e.g., impedance) based immunosensor 10 allows for the system 11 to use a single anti-calprotectin capture antibody 28 on working electrode 20 rather than multiple anti-calprotectin capture antibodies and/or anti-calprotectin detection antibodies, which may be required in other non-impedance based immunosensors. For example, sample solution 40 consists essentially of the calprotectin biomarker 32 and does not contain any other anti-calprotectin capture and/or anti-calprotectin detection antibodies intended to interact with immunosensor 10 and/or calprotectin biomarker 32. By excluding additional anti-calprotectin capture and/or anti-calprotectin detection antibodies from sample solution 40, sample solution 40 may be easily prepared by the patient or a caregiver. Also, any impact on the detectability or readability of the concentration of the calprotectin biomarker 32 in the sample solution 40 caused by additional anti-calprotectin capture and/or anti-calprotectin detection antibodies can be eliminated. Thus, this system 11 lowers the cost, complexity, and testing time associated with the testing of sample solutions 40 with immunosensor 10 as compared to other immunosensors that do not utilize impedance-based analysis. The use of an impedance-based immunosensor 10 can also detect calprotectin biomarker 32 concentrations in the sample solution 40 at higher ranges than other immunosensors because there is no need for additional dilution of the sample solution with a second anti-calprotectin capture and/or anti-calprotectin detection antibody.

System 11 includes measuring component 42. Measuring component 42 may be a physical measuring device that includes voltage source 46 and ammeter 44. Voltage source 46 may receive a voltage signal 52 from the processor 48 where voltage source 46 maintains a constant voltage between the reference electrode 16 and the working electrode 20. When either the calibration solution 39 or the sample solution 40 is introduced into the aperture 22, the sample solution 40 completes a circuit between all three electrodes 16, 18, 20. During the electrochemical analysis of the each solution 39 and 40, the constant voltage is supplied to both the working electrode 20 and the reference electrode 16 by voltage source 46. This constant voltage causes an electrical potential difference between the counter electrode 18 and working electrode 20, and the potential difference initiates electrical current to flow between the two electrodes 18 and 20. In the case of the sample solution 40, the extent of the reaction of the calprotectin biomarker 32 and the anti-calprotectin capture antibody 28 affects the current flowing between the two electrodes 18 and 20 by the buildup of resistance to current flow at the outer surface 26 of the working electrode 20. Ammeter 44 is connected to both working electrode 20 (e.g., via lead 36 and voltage source 46) and counter electrode 18 (e.g., via lead 34) and measures this current. Ammeter 44 outputs the measured current data 54 to processor 48 where the processor 48 determines the concentration of the calprotectin biomarker 32 in the sample solution 40 based upon the measured current data 54 (e.g., by ammeter 44), as described further below.

Processor 48 includes input/output module 50 and conversion module 56. Both input/output module 50 and conversion module 56 are used by processor 48 during the electrochemical analysis of the sample solution 40 to determine the concentration of calprotectin biomarker 32 in the sample solution 40. In some cases, processor 48 is a dedicated integrated circuit of analyzer 41 (e.g. a processor circuit) that processes the chemical analysis of each solution 39 and 40 by logic hard-wired into the circuitry of processor 48. In other examples, processor 48 is part of a more complex computational system, such as a central processing unit (CPU) of a general purpose computer that communicates with analyzer 41. In this case, the chemical analysis of the sample solution 40 is performed by the arithmetic logic unit (ALU) of processor 48, which is based upon executable instructions stored in the memory of the CPU of processor 48.

Input/output module 50 controls the voltage signal 52 outputted to voltage source 46 as well as receives the measured current data 54 from ammeter 44. Both the current data 54 and voltage signal 52 are used by processor 48 to determine the concentration of the calprotectin biomarker 32 in the sample solution 40.

Conversion module 56 includes circuit calculation component 58 and correlation component 68. Circuit calculation component 58 calculates an impedance measurement (Z) by dividing the voltage signal 52 (V) by the current data 54 (I) according to Ohm’s Law and then calculates R_{ct} 62 according to a representative electrical circuit 59, which is an example of a modified Randle’s equivalent electrical circuit. The representative circuit includes both capacitive components CPE₁ 64 and CPE₂ 66, as well as resistive components R_{S} 60 and R_{ct} 62. These calculations are represented by the following formula:

$$Z = \frac{V}{I} = {R}_{S} + \frac{1}{\frac{1}{{R}_{c t}} + j ω C P {E}_{1}} + \frac{1}{j ω C P {E}_{2}}$$

In the case where the working electrode 20 is a screen printed gold electrode, constant capacitive elements CPE₁ 64 and CPE₂ 66 are used due to the rough surface of the working electrode 20 having a large surface area for biomolecule functionalization. R_{S} 60 corresponds with the ohmic resistance of the electrolyte in the solution 39, 40, and is also considered a constant value because the ohmic resistance of the solution 39, 40 does not significantly change based upon functionalization of the anti-calprotectin capture antibody 28 to the working electrode 20. As noted above, R_{ct} 62 corresponds with the charge transfer resistance at the outer surface 26 of the working electrode 20.

Circuit calculation component 58 shares R_{ct} 62 with correlation component 68, which associates R_{ct} 62 with the concentration of the calprotectin biomarker 32 based upon a predetermined configurable relationship, specifically a predetermined configurable linear relationship 70 between R_{ct} 62 and the concentration of the calprotectin biomarker 32. The predetermined linear relationship 70 of FIG. 2 is represented by the following formula:

$$Δ {\text{R}}_{\text{ct}} \left(Ω\right) = \text{B} \times \left[\text{CP}\right] + \text{A}$$

In this formula, ΔR_{ct} may be the difference between the actual R_{ct} 62 when exposed to the sample solution 40 and the calibrated R_{ct} when exposed to the calibration solution 39. [CP] is the concentration of calprotectin biomarker 32 in the sample solution 40, and B and A are constants attained through experimentation. Due to imperfections and irregularities in manufacturing, each working electrode 20 may be slightly different and may exhibit a slightly different resistance response to calprotectin. The calibration solution 39 may compensate for this variability.

Once processor 48 has calculated the concentration of calprotectin biomarker 32 in the sample solution 40, the results are outputted to results device 72, which may display the concentration of the calprotectin biomarker 32 in the sample solution 40 to a user (e.g., a doctor, patient, technician, etc.). As described with reference to FIG. 1 above, the concentration of the thiolated calprotectin antibodies (e.g., the anti-calprotectin capture antibody 28) on the outer surface 26 of the working electrode 20 allows for a wide range of detectability of the calprotectin biomarker 32 in the sample solution 40. In this case, system 11 is able to detect the concentration of the calprotectin biomarker 32 in the sample solution 40 from 4 ng/ml to 240 ng/ml, which corresponds to a measured a concentration of 30 µg/ml to 1800 µg/ml of calprotectin biomarker in the raw sample solution as diluted in a typical ELISA protocol. The detected concentration range corresponds with a higher detectable range of calprotectin than other immunosensors. In other cases, system 11 may also able to detect the concentration of the calprotectin biomarker 32 in the sample solution 40 at either higher and/or lower concentration ranges than 4 ng/ml to 240 ng/ml. In either case, the detected calprotectin biomarker 32 concentration may be used to differentiate between IBD and irritable bowel syndrome (IBS). The detected calprotectin biomarker 32 concentrations less than 50 µg/ml may correspond to little to no inflammation, concentrations of 50 µg/ml to 150 µg/ml may correspond to mild inflammation, and concentrations greater than 150 µg/ml may correspond to organic inflammation (e.g., IBD) in GI.

The immunosensor 10 may be a single-use device. Thus, after detecting the concentration of the calprotectin biomarker 32, the immunosensor 10 may be discarded.

### EXAMPLES

Experimental examples are presented herein where: in a first experimental example, the fabrication of a working electrode was studied, in a second experimental example, the impact of a working electrode’s construction on electrochemical performance was studied, in a third experimental example, the impact of a working electrode immobilized antibody concentration on electrochemical performance was studied, in a fourth experimental example, the impact of calprotectin concentration on the electrochemical performance was studied, in a fifth experimental example, the specificity of the immunosensor was studied, in a sixth experimental example, the practical application of the immunosensor was determined, and in a seventh experimental example, the performance of the immunosensor was compared with ELISA. Each of the experimental examples are meant to provide further clarification of how immunosensor 10 was either fabricated or tested when developing the immunosensor 10 and system 11. Any information provided herein is meant to support the prior disclosure and additionally provide non-limiting features and/or descriptions via the specific experimental discussions herein.

### Example 1: Fabrication of Working Electrode

In the first experimental example, the fabrication of the working electrode 20 was studied. The immunosensor was fabricated by the following process: prior to functionalization of SPGE with CPAb (mouse monoclonal anti-calprotectin antibody), equal volumes of thiolation reagent and 1 mg/ml concentrations of CPAb is mixed and incubated at room temperature for 1 hour to add thiol groups. This modifies amines to sulfhydryl which form thioether linkage with other molecules. 10 µL (5 µg/ml) of thiolated CPAb were drop cast on SPGE and are incubated overnight at 4° C. The attached antibodies provide a stable high-affinity surface for selective binding of calprotectin to the gold electrode surface (e.g., a 5 mm working electrode diameter). Then the electrodes were washed with 1X PBS to remove any unbound CPAb. To minimize non-specific binding the sites of the immunosensor were blocked with 1XPBS containing 5X BSA for 1 hour at 37° C. and labelled as SPGE-CPAb-BSA.

### Example 2: Impact of Working Electrode Construction on Electrochemical Performance

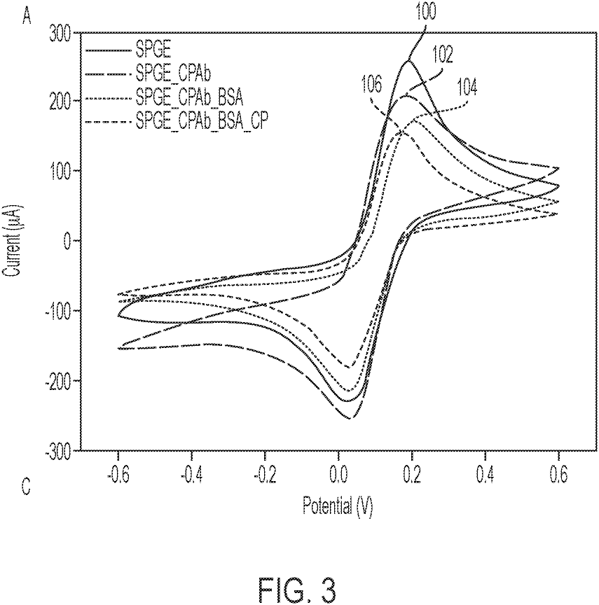

In a second experimental example, the impact of a working electrode’s construction on electrochemical performance was studied. Electrochemical measurements were carried out using a Gamry instrument (Reference 3000 Potentiostat/Galvanostat/ZRA) controlled by framework data acquisition software (Version 6.23). All measurements were performed in a background solution of 10 mM K₃Fe(CN)₆/K₄Fe(CN)₆ (1:1) in 1X PBS (10 mM, Phosphate buffered saline) (pH 7.4) at room temperature (25° C.). A typical three-electrode system containing gold (5 mm diameter), platinum and Ag/AgCl as working, counter and reference electrodes respectively is found in SPGE. Cyclic voltammetry (CV) scans were measured in a potential window of -0.5 to 0.5 V at 100 mV/s scan rate. EIS of bare SPGE and modified SPGE were analyzed with an input potential of 50mV amplitude that scanned over the 1-100,000 Hz frequency range with an increment of 10 frequencies per decade. The impedance spectral analysis by an equivalent circuit model was done using a non-linear curve fitting software (Gamry analyst). The change in electrochemical behavior of SPGE at different stages of modification by immune species was determined by these CV scans, as illustrated in FIG. 3. To realize this, CV scans were performed using a solution of 10 mM K₃Fe(CN)₆/K₄Fe(CN)₆ in 1 X PBS. The voltagram of bare SPGE show expected oxidation and reduction peaks with a peak 100 current of 258 ± 10.981 µA. After functionalizing SPGE with 10 µl of 5 µg/ml anti-calprotectin antibody (SPGE_CPAb), BSA (SPGE_CPAb_BSA), and calprotectin (SPGE_CPAb_BSA_CP) the peak current reduced to peak 102: 208 ± 9.8736, peak 104: 178 ± 18.210, and Peak 106: 156.92 ± 15.649 µA respectively. The relative decrease in the peak current correspond to suggests that immune species functionalized on SPGE contributed an insulating layer which in turn hindered the transport of [Fe(CN)6]3-/4- ion towards SPGE. Ferricyanide response was reduced after the addition of CPAb and CP to SPGE. This showed enlarged peak-to-peak separation in the cathodic and anodic regions when compared to bare SPGE. This further confirms that biomolecules are immobilized onto the electrode surface.

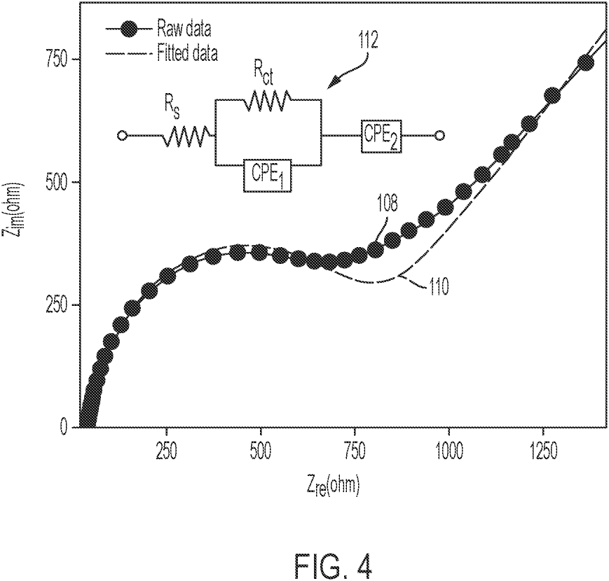

The interfacial properties of the immunosensor (SPGE_CPAb_BSA_CP) was studied by fitting impedance data using a modified Randle’s equivalent circuit 112, as illustrated in FIG. 4. The measured (Dotted) line 108 and fitted (solid) line 110 impedance spectrum are shown in FIG. 4 revealing fitting over the measured frequency range. The modified circuit 112 has four elements. The SEM images of SPGE have shown the SPGE to have a rough surface, and therefore, a large surface area for biomolecule functionalization. In this case, constant phase element (CPE₁ & CPE₂) are used instead of classical capacitance in the equivalent circuit. Here CPE₁ and CPE₂ represent electrolyte and electrode sides of the interface. The last element in the equivalent circuit is charge transfer resistance (R_{ct}). The total current through the working interface is the sum of the faradaic process and double layer capacitance hence the elements CPE₁ and CPE₂+R_{ct} were introduced parallelly in the equivalent circuit 112. The ohmic resistance of the solution appears not to change with bio-functionalization on SPGE. However, R_{ct} increased relatively after each stage of biomolecule functionalization it acted as an inert blocking layer for electron and mass transfer. This phenomenon hindered the diffusion of ferricyanide ions towards the electrode surface.

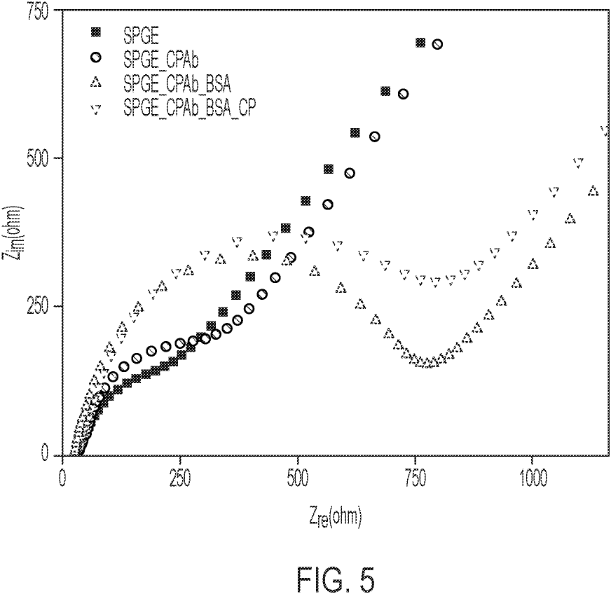

In FIG. 5 the faradaic impedance spectra of biomolecule functionalized of SPGE are shown. R_{ct} value of SPGE was found to be 241.21±17.6472 ohms which increased with different stages of biomolecule functionalization. After the addition of CPAb (SPGE_CPAb), BSA (SPGE_CPAb_BSA), and CP dissolved in incubation buffer (IB) (SPGE_CPAb_BSA_CP) the R_{ct} values increased to 348.78±11.8190, 580.5±11.6 and 602.8 ± 16.17 ohms, respectively. Especially after the addition of BSA and CP, the semicircle size increased drastically, as these are negatively charged at pH of 7.4 and potentially blocked most of the exposed surfaces on SPGE. Further, after the addition of calprotectin R_{ct} increased confirming the detection mechanism of the immunosensor.

### Example 3: Impact of Immobilized Antibody Concentration on Electrochemical Performance

In a third experimental example, the impact of the working electrode immobilized with differing antibody concentration on electrochemical performance was studied. In this case, the biosensor interface (CPAb) concentration was varied 0, 1, 5, 10, 20 and 100 µg/ml to attain electrochemical signal in response to increasing concentration of CP. The calibration curve was obtained by exposing the immunosensors to varying concentrations of CP of 4, 12, 40, 120, and 240 ng/ml corresponding to the extended working range of 30, 90, 300, 900 and 1800 µg/ml from an ELISA kit.

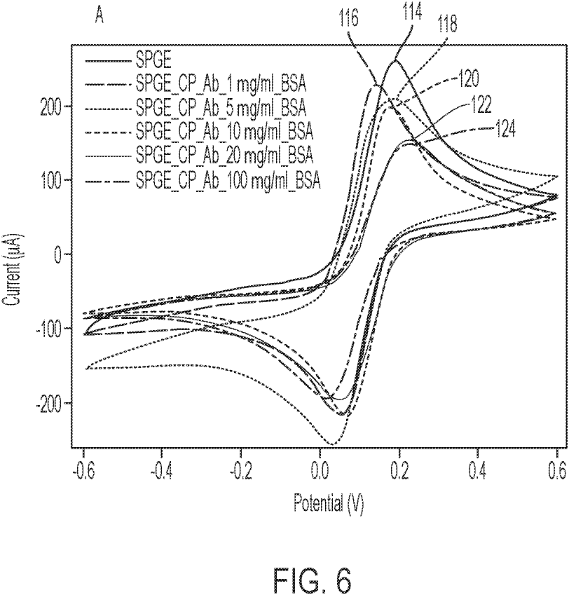

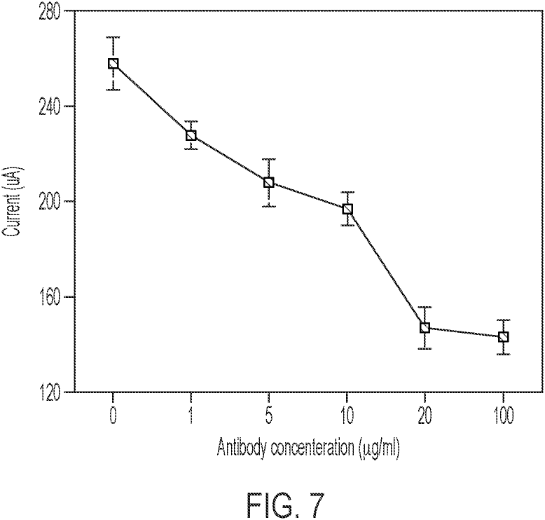

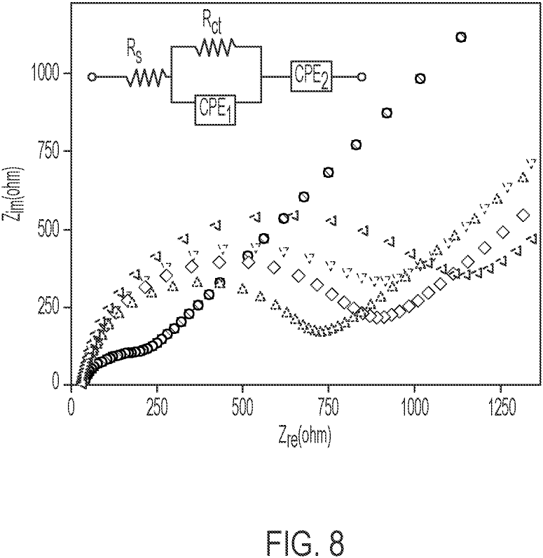

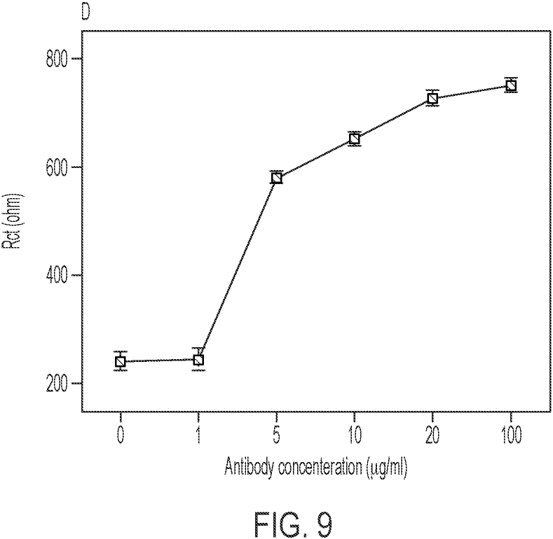

To determine the optimal electrochemical signal in response to varying concentrations CP, the concentrations of biosensor interface was optimized. As the antibody coating on the immunosensor aids in specific binding of the respective antigen, the concentration of the antibody on the immunosensor is important in determining the immunosensor performance. Both CV and EIS responses of the SPGE at different concentrations of anti-calprotectin antibody immobilized electrodes were recorded. FIG. 6 shows the voltagram and peak currents of SPGE_CPAb_BSA ( and 100 µg/ml). The peak currents were peak 114: 258±10.981, peak 116: 228±5.671, peak 118: 208±9.8736, peak 120: 197±6.971, peak 122: 147±8.731, and peak 124: 143±7.2019 respectively. As illustrated in FIG. 7, the reduction in peak current was due to the increased insulating effect contributed by increased concentrations of CPAb. FIG. 8 shows the EIS response and its respective R_{ct} values of SPGE_CPAb_BSA (0, 1, 5, 10, 20 and 100 µg/ml). The R_{ct} values are 241.21±17.64, 244.4±20.55, 580.5±11.6, 652.4±12.73, 727.3±14.61, and 750.91±12.21 respectively. The increase in R_{ct} values confirms the increased coverage of electrode surface in an insulating layer on the electrode surface. As illustrated in FIG. 9, reduction in peak current and increase in R_{ct} values followed a trend until approximately 20 µg/ml of CPAb after which negligible reduction was observed. These observations lead to the conclusion that 20 µg/ml of CPAb was enough to completely cover the sensing.

### Example 4: Combined Impact of Immobilized Antibody Concentration and Calprotectin Concentration on Electrochemical Performance

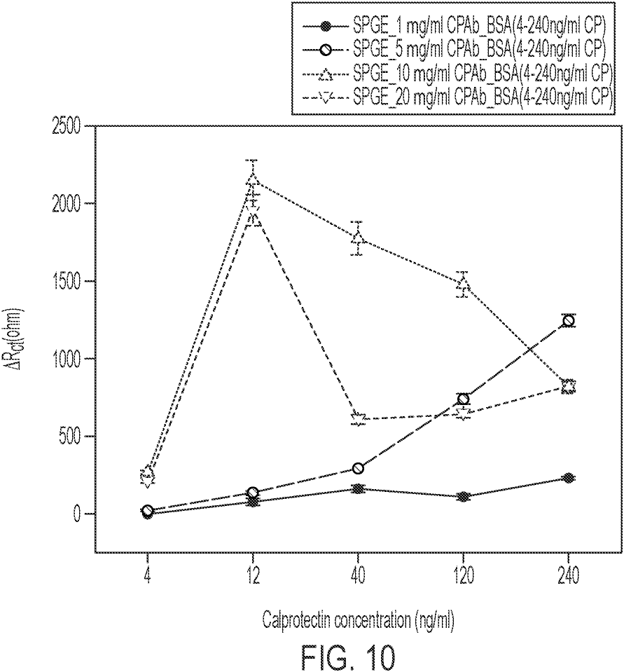

In a fourth experimental example, the impact of calprotectin concentration on electrochemical performance of the immunosensor was studied. In this case, a calibration curve was obtained by exposing the immunosensors to varying concentrations of CP 4, 12, 40,120, and 240 ng/ml corresponding to the extended working range of 30, 90, 300, 900 and 1800 µg/ml from an ELISA kit. In this case, CP sensing ability of SPGE_CPAb_BSA functionalized with 1, 5, 10, and 20 µg/ml was tested by the electrodes to varying concentration CP (4, 12, 40, 120 and 240 ng/ml). Δ R_{ct} = R_{ct} _{SPGE}_{_CPAb}_{_BSA}_{_CP} - R_{ct} _{SPGE}__{CPAb}_{_BSA} values were calculated and are illustrated in FIG. 10 where Δ R_{ct} represents change in charge transfer resistance caused by an increase in CP antigen coupled to the CPAb on the SPGE surface. Based on the results, the Δ R_{ct} values of SPGE_CPAb_BSA with different amounts of immobilized CPAb showed an overall increasing trend at 4 and 12 ng/ml. However, at higher concentrations of CP (40, 120 and 240 ng/ml) a sharp decline in Δ R_{ct} values of SPGE_CPAb_BSA functionalized with 10, and 20 µg/ml was observed. This may be due to overcrowding of the electrode’s surface with CPAb, and surpassed monolayer coverage which may favor the inaccessibility of antibodies by CP by overlaying effects. Reduction in active sites of CPAb causing hooked responses at very large CP concentration (40, 120 and 240 ng/ml). It could be concluded that at 5 µg/ml of CPAb is optimal to get a steady electrochemical response to sense a wide range of CP concentration.

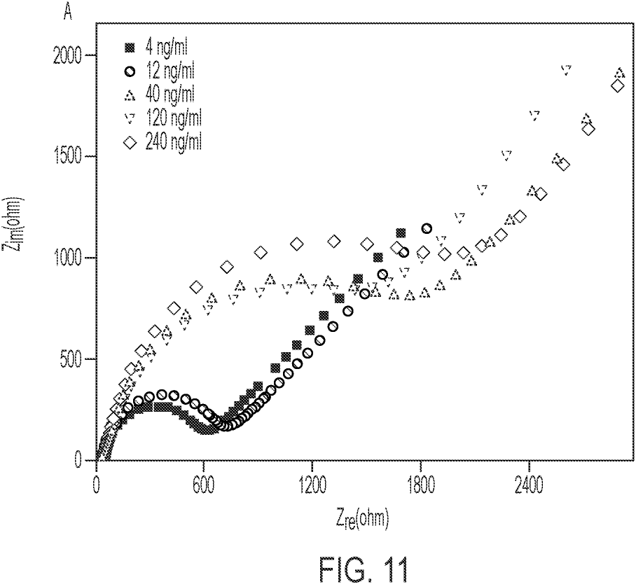

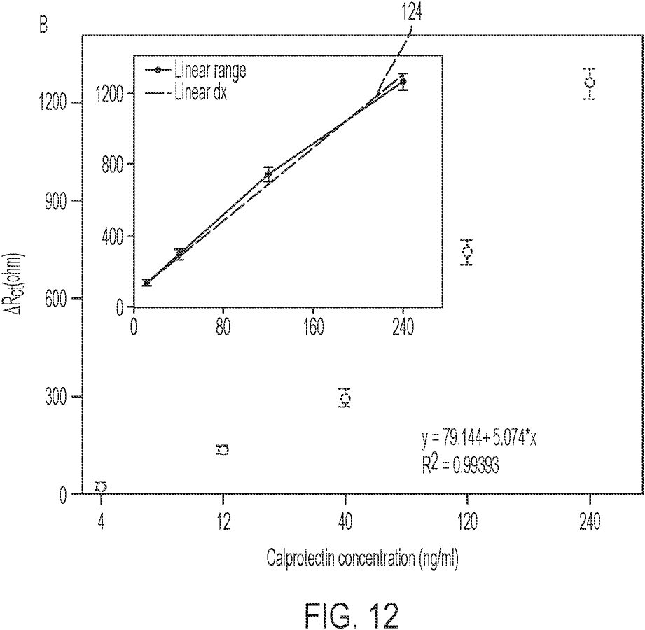

FIG. 11 shows the Nyquist plot of SPGE_CPAb-5 µg/ml _BSA exposed to 4, 12, 40, 120, and 240 ng/ml of CP. From this, it can be found that the diameter of the semicircle region increased with an increase in CP concentration. This may be due to the binding of higher CP molecules with the CPAb providing an effective insulating barrier for the ferricyanide redox probe. The respective Δ R_{ct} values were used to plot the calibration curve as shown in FIG. 12. A linear relationship 126 between CPAb and CP was observed in the range of 12 to 240 ng/ml of CP. The observed linear regression equation is ΔR_{ct} (Ω) = 79.144 + 5.074×CP with a correlation coefficient of 0.99393. Based on this CP concentration in each environment can be quantitatively measured. The developed immunosensor for CP displayed a well-defined concentration-response.

### Example 5: Specificity Testing in Presence of Other Inflammatory Proteins

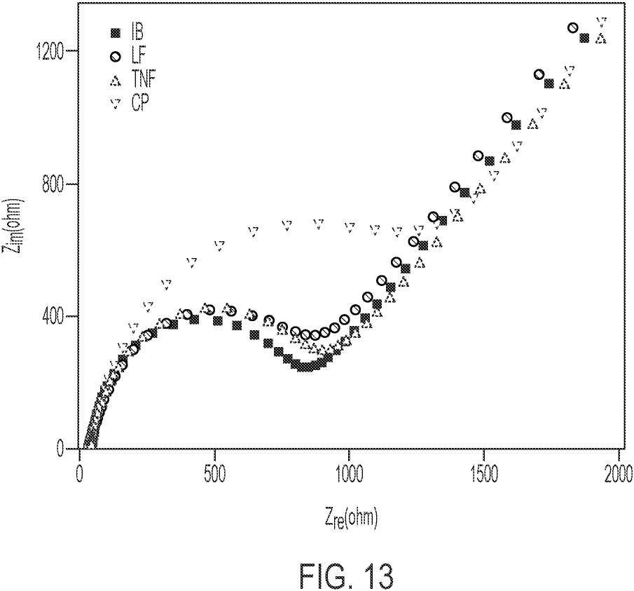

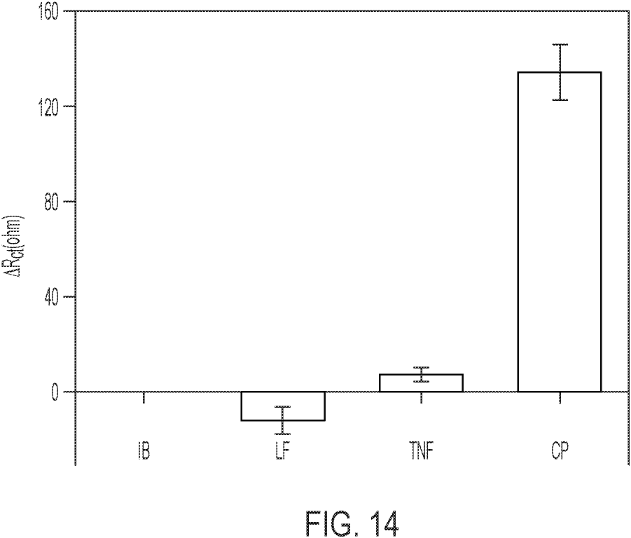

In a fifth experimental example, the specificity of the immunosensor was studied. In this case, an immunosensor functionalized with CPAb and blocked with BSA were exposed to 12 ng/ml of other inflammatory proteins such as lactoferrin (LF), Tumor necrosis factor (TNF). Along with these competing proteins 12 ng/ml of calprotectin (CP) and incubation buffer (IB) were included in this study. As illustrated in FIG. 13, EIS response of the mentioned analytes revealed that the Δ R_{ct} values of IB, LF, TNF and CP to be 0, -12.14 ± 5.7, 7.3 ± 2.89, 134.6 ± 11.6 respectively. As illustrated in FIG. 14, in the presence of other analytes like LF, TNF, and IB no significant change in R_{ct} values was observed. This confirmed the selective binding properties of the developed immunosensor.

### Example 6: Relationship Between Resistance and Calprotectin Concentration

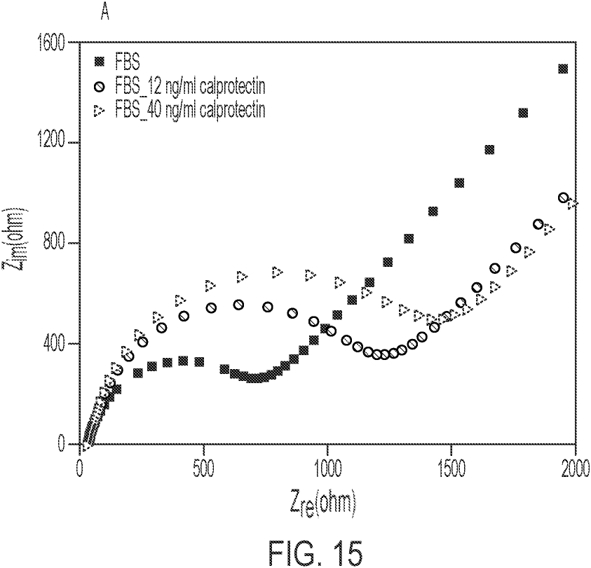

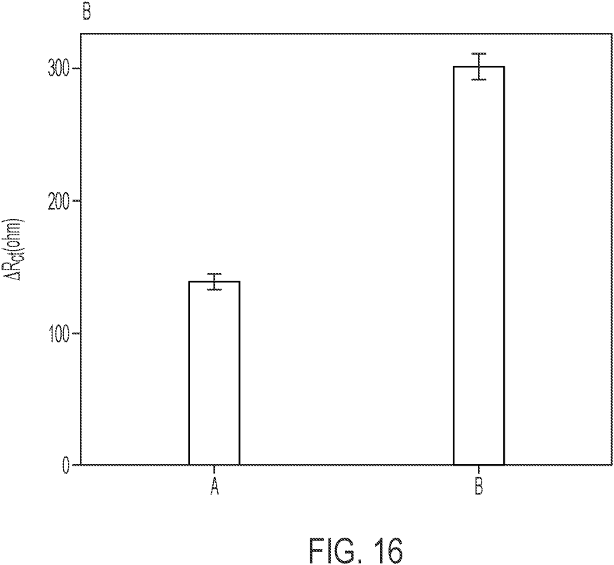

In a sixth experimental example, the practical application of an immunosensor was validated by detecting CP spiked in FBS. The presence of CP in complex samples was determined by exposure to FBS spiked with calprotectin. FIG. 15 shows a Nyquist plot showing the impedance response of the immunosensor (SPGE_CPAb_BSA) exposed to FBS alone and FBS spiked with 12 and 40 ng/ml of CP. R_{ct} values of FBS exposed immunosensor are similar SPGE_CPAb_BSA. However, CP in FBS induced a sequential increase in the diameter of the semi-circle region of impedance spectrum was observed and its ΔR_{ct} values were 138.65 ± 5.6421 and 301.2 ± 9.69854 as illustrated in FIG. 16.

The observed electrochemical response obtained is due to CP concentration and not because of FBS shows the applicability of the immunosensor with real samples. This sensor can potentially be used as a complementary approach for routine lab analysis of CP.

### Example 7: Comparative Example to ELISA

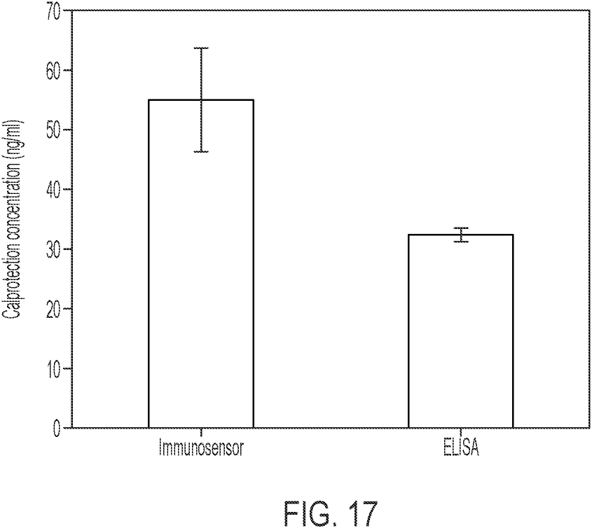

In a seventh experimental example, the performance of the immunosensor was compared with ELISA. In this case, 40 ng/ml of CP dissolved in incubation buffer was quantified by immunosensor and ELISA, and is shown in FIG. 17. The concentration measured by the techniques was 54.91 ± 8.654 and 32.41 ± 1.15 respectively. The relative standard deviation percentage between two techniques was found to 3.36% (ng/ml). These results show that the immunosensor performance is in agreement with ELISA which is the current clinical method for CP quantification. Hence the immunosensor could be used in a clinical setting for point of care diagnosis of CP.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

1. An electrochemical biosensor for detection of a calprotectin antigen in a sample solution, the electrochemical biosensor comprising:
a reference electrode;
a counter electrode; and
a working electrode comprising a surface coated with an anti-calprotectin antibody configured to bind to the calprotectin antigen in the sample solution; and
wherein the biosensor is configured to detect a concentration of the calprotectin antigen in the sample solution based on a resistance change at the surface of the working electrode.

2. The electrochemical biosensor of claim 1, wherein the reference electrode, the counter electrode, and the working electrode are formed on an electrode layer on the surface of a substrate.

3. The electrochemical biosensor of claim 1, wherein the anti-calprotectin antibody comprises a thiolated mouse monoclonal anti-calprotectin antibody.

4. The electrochemical biosensor of claim 1, wherein the surface of the working electrode is coated with an antibody solution of the anti-calprotectin antibody.

5. The electrochemical biosensor of claim 4, wherein the concentration of the anti-calprotectin antibody in the antibody solution is 1 µg/mL to 20 µg/mL.

6. The electrochemical biosensor of claim 4, wherein the concentration of the anti-calprotectin antibody in the antibody solution is 4 µg/mL to 6 µg/mL.

7. The electrochemical biosensor of claim 4, wherein the concentration of the anti-calprotectin antibody in the antibody solution is 4.5 µg/mL to 5.5 µg/mL.

8. The electrochemical biosensor of claim 4, wherein the concertation of the anti-calprotectin antibody in the antibody solution is 5 µg/mL.

9. The electrochemical biosensor of claim 4, wherein the antibody solution consists of the anti-calprotectin antibody and a bovine serum.

10. The electrochemical biosensor of claim 1, wherein the biosensor detects the concentration of the calprotectin antigen in the sample solution from 4 ng/mL to 240 ng/mL.

11. A method of determining a concentration of a calprotectin antigen in a sample solution with a biosensor, the method comprising:
providing the biosensor comprising a reference electrode, a counter electrode, and a working electrode, the working electrode having a surface coated with an anti-calprotectin capture antibody;
providing the sample solution consisting essentially of the calprotectin antigen and lacking an anti-calprotectin detection antibody;
exposing the sample solution to the biosensor such that the calprotectin antigen binds to the anti-calprotectin capture antibody on the working electrode;
measuring a current between the counter electrode and the working electrode; and
determining the concentration of the calprotectin antigen in the sample solution based upon the measured current.

12. The method of claim 11, wherein the current decreases as the concentration of the calprotectin antigen in the sample solution increases.

13. The method of claim 11, wherein the surface of the working electrode is coated with an antibody solution of a thiolated form of the anti-calprotectin capture antibody at a concentration of 4 µg/ml to 6 µg/ml.

14. The method of claim 13, wherein the concentration of the anti-calprotectin capture antibody in the antibody solution is 4.5 µg/ml to 5.5 µg/ml.

15. The method of claim 11, wherein the concentration of the calprotectin antigen in the determining step is 4 ng/mL to 240 ng/mL.

16. A system for detecting a concentration of a calprotectin antigen in a sample solution, the system comprising:
an electrochemical biosensor comprising a working electrode, a reference electrode, and a counter electrode, wherein an outer surface of the working electrode is coated with an anti-calprotectin antibody configured to bind to the calprotectin antigen in the sample solution; and
an analyzer in electrical communication with the biosensor and configured to determine a concentration of the calprotectin antigen in the sample solution from 4 ng/mL to 240 ng/mL based upon an electrical current flowing between the working electrode and the counter electrode.

17. The system of claim 16, wherein the outer surface of the working electrode is coated with a solution of the anti-calprotectin antibody at a concentration of 4.5 µg/ml to 5.5 µg/ml.

18. The system of claim 17, wherein the outer surface of the working electrode is coated with a 5 µg/ml solution of the anti-calprotectin antibody.

19. The system of claim 16, wherein the analyzer is configured to associate the electrical current with a resistance change on the outer surface of the working electrode, and to correlate the resistance change with the concentration of the calprotectin antigen in the sample solution.

20. The system of claim 16, wherein the working electrode is coated with a single type of the anti-calprotectin antibody.
